(19) United States
(12) Patent Application Publication
(10) Pub. No.: US 2014/0134070 A1
(43) Pub. Date: May 15, 2014

(54) Method and Apparatus for Sanitizing a Writing Instrument

(71) Applicant: Wendy Owens, Auburndale, FL (US)

(72) Inventor: Wendy Owens, Auburndale, FL (US)

(21) Appl. No.: 13/672,803

(22) Filed: Nov. 9, 2012

(51) Int. Cl. A61L 2/18

(52) U.S. Cl. CPC A61L 2/18
USPC 422/292

(57) ABSTRACT

An apparatus for sanitizing a writing instrument comprising a vessel capable of containing an absorbent material, a sanitizing fluid and a writing instrument. The apparatus includes a cover, capable of being attached to the vessel and having an aperture through which a writing instrument may be received. An absorbent material is disposed within the vessel, and includes a channel for receiving a writing instrument for sanitizing. A sanitizing fluid capable of sanitizing a writing instrument, is disbursed within the absorbent material. A writing instrument is attached to the apparatus with a connector and can be inserted into the aperture and further inserted into the channel of the absorbent material. When inserted into the absorbent material, the writing instrument is sanitized by the sanitizing fluid disbursed within the absorbent material.

### FIELD OF THE INVENTION

The subject invention pertains to the field of methods and apparatus for sanitization, more particularly to methods and apparatus for sanitizing writing instruments.

### BACKGROUND OF THE INVENTION

Writing instruments attached to counters in business such as banks and the like are breading grounds for pathogens. Constant use by patrons leaves a person at risk to the hygiene habits of complete strangers. The use of disinfectants and other cleaners provides only a limited amount of protection as the surface of such instruments would require constant cleaning to avoid transmission of biologic pathogens. Dangerous pathogens such as Influenza, Staphylococcus aureus, Streptococcus pneumoniae, Escherichia coli, methicillin-resistant Staphylococcus aureus (“MRSA”) Salmonella spp., Campylobacter spp., Listeria spp., Trichinella spp., Shigella sp., Bacillus spp. and various viral pathogens can be found on writing instruments and substrates.

Accordingly, a writing instrument tethered to a counter in a businesses can provide an excellent vector for the exchange of such pathogens due to the number of persons who use the writing instrument in a short period of time and the unsanitary habits of the prior people who have used the writing instrument. Prior art solutions to the aforementioned issues are generally limited to the direct application of disinfectant solutions. Some disinfectant solutions can contain caustic or dangerous chemicals that should not come into contact with human skin.

The documents and publications cited in this disclosure are incorporated herein by reference in their entirety, to the extent they are not inconsistent with the explicit teachings set forth.

### BRIEF SUMMARY OF THE INVENTION

An apparatus for sanitizing a writing instrument includes a vessel, a cover, an absorbent material, a sanitizing fluid and a writing instrument. The vessel can be capable of containing the absorbent material, sanitizing fluid and the writing instrument. The cover can be capable of being attached to the vessel and can have an aperture through which the writing instrument may be received. The absorbent material can be disposed within the vessel and can comprise a channel for receiving a writing instrument for sanitizing. The sanitizing fluid can be disbursed within the absorbent material and can be capable of sanitizing the writing instrument and in the vessel.

The writing instrument can be attached to said apparatus with a connector. The writing instrument can also be inserted into the aperture and further inserted into the channel in the absorbent material. The writing instrument can be sanitized by the sanitizing fluid disbursed within the absorbent material.

The vessel cab be constructed from a material resistant to degradation by said sanitizing fluid. The aperture in the cover can be concentric to the channel in the absorbent material. It is preferred that the width of the channel in the absorbent material is smaller than the width of said writing instrument.

The absorbent material can be manufactured from a material, or materials including: sponge, cloth, natural material, synthetic material, wool, grass, hemp, sisal, straw, bamboo, cotton, linen, flax, jute, felt, casein fiber, silk, velvet, taffeta, lyocell, glass fiber, polyester, polyurethane, aramid, acrylic, acetate, nylon, spandex, olefin fiber, carbon fiber, burlap, and canvas.

The writing instrument contemplated in this disclosure can include a pen, a pencil, a marker or any other apparatus capable of being used for writing. The writing instrument contemplated herein can be resistant to degradation by the sanitizing fluid. As contemplated herein, the sanitizing fluid can be hypoallergenic.

The connector disclosed herein can be resistant to degradation by said sanitizing fluid. Connectors contemplated herein include chain, rope, string, straps, webbing, cord, cable, monofilament line, woven line, string or any other similar devices capable of operating as a connector.

The bottom of the apparatus disclosed herein can include a means to maintain the position of the apparatus on a substrate. By way of example, such means can include, a non-slip surface, additional weight, a suction cup, or any other means known in the art to maintain the position of the apparatus on a substrate.

Accordingly, it is an object of the present invention to provide a sanitizing apparatus.

It is a further object of the present invention to provide a sanitizing apparatus for writing instruments.

Further objects and advantages of the present invention will become apparent by reference to the following detailed disclosure of the invention and appended drawings wherein like reference numerals refer to the same feature, component or element.

### BRIEF DESCRIPTION OF THE DRAWINGS

FIG. 1 is a perspective view of an apparatus for sanitizing writing instruments according to aspects of the present invention.

FIG. 2 is a perspective view of an uncovered apparatus for sanitizing writing instruments according to aspects of the present invention.

FIG. 3 is a perspective view of the cover of an apparatus for sanitizing writing instruments according to aspects of the present invention.

FIG. 4a is a side view of the bottom of an apparatus for sanitizing writing instruments according to aspects of the present invention.

FIG. 4b is a side view of the bottom of an apparatus for sanitizing writing instruments according to aspects of the present invention.

FIG. 5 is a side view of an alternative embodiment of an apparatus for sanitizing writing instruments according to aspects of the present invention.

### DETAILED DISCLOSURE OF THE INVENTION

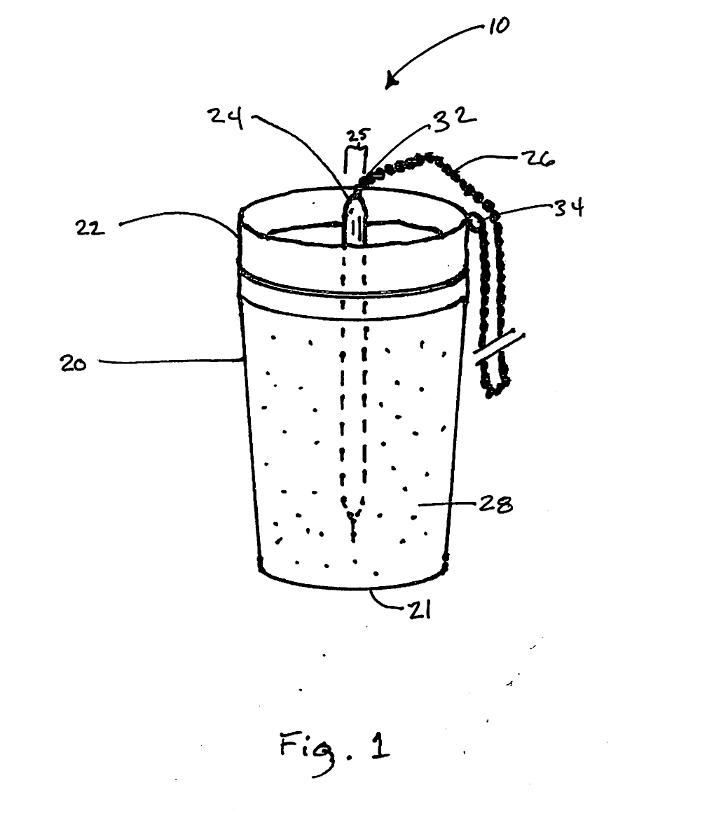

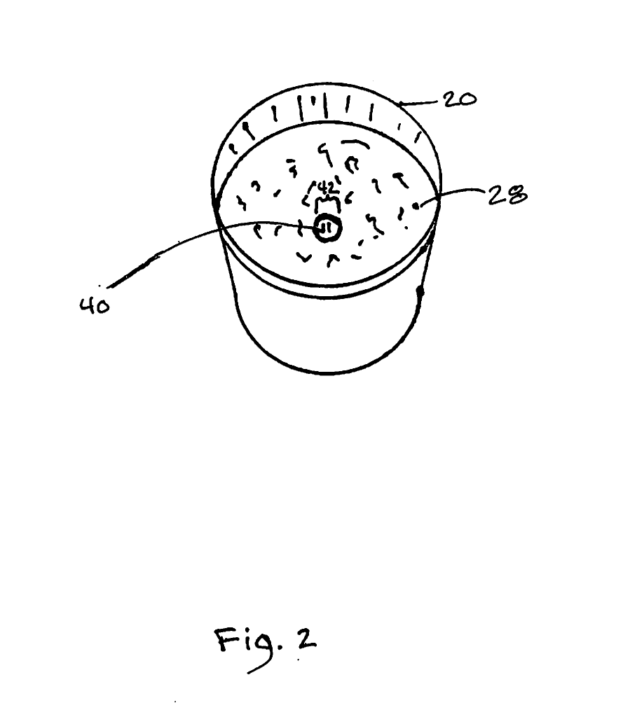

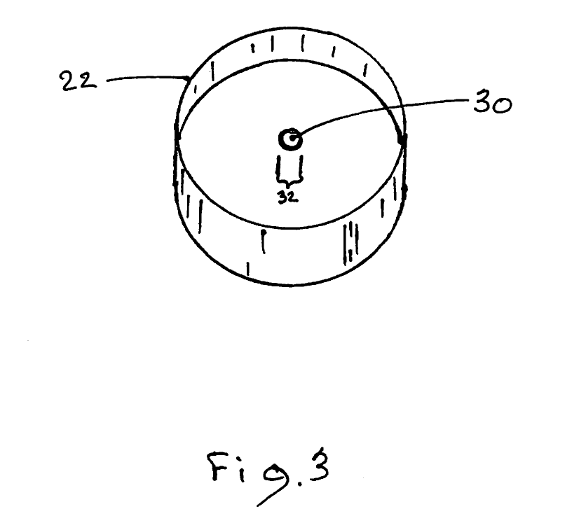

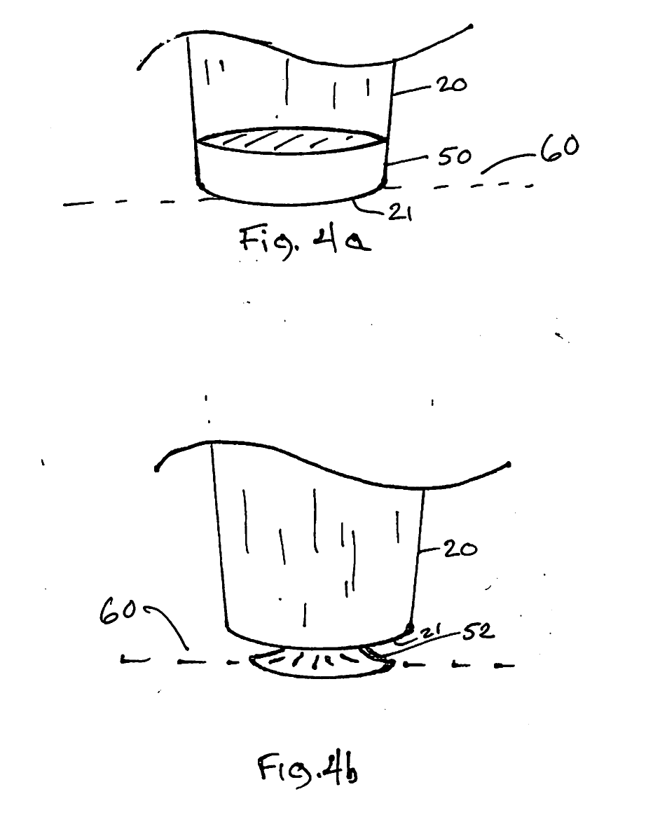

Referring now to FIG. 1 the apparatus according to the present invention is illustrated and generally referred to by reference numeral 10.

The apparatus for sanitizing a writing instrument comprises a vessel 20 further comprising a cover 22, wherein the vessel 20 is capable of holding sanitizing fluid (not shown), an absorbent material 28 and a writing instrument 24. In a preferred embodiment, the vessel 20 is generally cylindrical and of a sufficient size to receive a substantial portion of a writing instrument 24 introduced therein. In addition, it is preferred that the vessel 20 be manufactured from a material resistant to degradation from any chemicals in the sanitizing fluid. Materials contemplated herein include metal, glass, plastic, ceramic, porcelain, or any other material capable of maintaining structural integrity when exposed to a sanitizing fluid.

The cover 22 can comprise an aperture 30 located in a substantially central location on the cover 22 through which a writing instrument 24 may be inserted. Accordingly, the width 32 of the aperture 30 in the cover 22 must be larger than the width 25 of the writing instrument 24. The cover 22 can be removably attached to the vessel 20 to facilitate the introduction or re-filling of sanitizing fluid into the vessel 20. The cover 22 can be attached to the vessel 20 using threads (not shown), clamps(not shown), o-rings(not shown), friction(not shown), seals(not shown), or any other method known in the art. In a preferred embodiment, when connected to the vessel 20, the cover 22 comprises a liquid-tight seal through which a sanitizing fluid cannot pass.

The absorbent material 28 can be sponge, or the like that can take the shape of, and maintain position inside, the vessel 20. Additional embodiments contemplate that the absorbent material 28 can comprise cloth, natural material, synthetic material, wool, grass, hemp, sisal, straw, bamboo, cotton, linen, flax, jute, felt, casein fiber, silk, velvet, taffeta, lyocell, glass fiber, polyester, polyurethane, aramid, acrylic, acetate, nylon, spandex, olefin fiber, carbon fiber, burlap, canvas and any other absorbent material capable of having a sanitizing fluid disbursed therein. In a preferred embodiment, the absorbent material 28 is comprised of a material capable of being in near constant contact with a sanitizing fluid and absorbent material 28 without being degraded by the chemicals in the sanitizing fluid.

The absorbent material 28 can further comprise a generally cylindrical channel 40 for receiving and maintaining a writing instrument 24. In a preferred embodiment, the width 42 of the channel 40 is smaller than the width 25 of the writing instrument 24. It is contemplated herein that the channel 40 in said absorbent material 28 is concentric with the aperture 30 in the cover 22.

The sanitizing fluid (not shown) can comprise a surface sanitizer, such as, for example a commercially available hypoallergenic sanitizer. In a preferred embodiment, the sanitizing fluid is not caustic to humans. In addition the sanitizing fluid can be fast drying to avoid unnecessary liquid on documents when the writing instrument is used.

The writing instrument 24 can comprise any writing apparatus, such as, for example, a pen, a pencil, or a marker. In a preferred embodiment, the writing instrument 24 is capable of being in near constant contact with a sanitizing fluid and absorbent material 28 without being degraded by the chemicals in the sanitizing fluid. In addition, it is preferred that the writing instrument 24 is capable of being in near constant contact with the sanitizing fluid and absorbent material 28 without leaking ink. By way of example, the Fisher Space Pen® is a writing apparatus that is resistant to leakage when exposed to liquids.

In alternative embodiments, the apparatus 10 can further comprise a connector 26 for connecting said writing instrument 24 to said apparatus 10 wherein said connector 26 is attached at a first end 34 to said apparatus 10 and attached at a second end 36 to said writing instrument 24. The connector 26 can be connected to the apparatus 10 on a location on the vessel 20 or on the cover 22. It is contemplated herein that the connector can be manufactured from chain, rope, string, straps, webbing, cord, cable, monofilament line, woven line, string or any other material capable of connecting the writing instrument 24 to the vessel 20.

The bottom 21 of the vessel 20 can further comprise a means for maintaining the position of the vessel 20 on a substrate 60. Such means can include a non-slip surface (not shown), double-sided tape (not shown), adhesive (not shown), additional weight 50, at least one suction cup 52, or any other apparatus known in the art for maintaining stability of the apparatus 10 on a substrate 60.

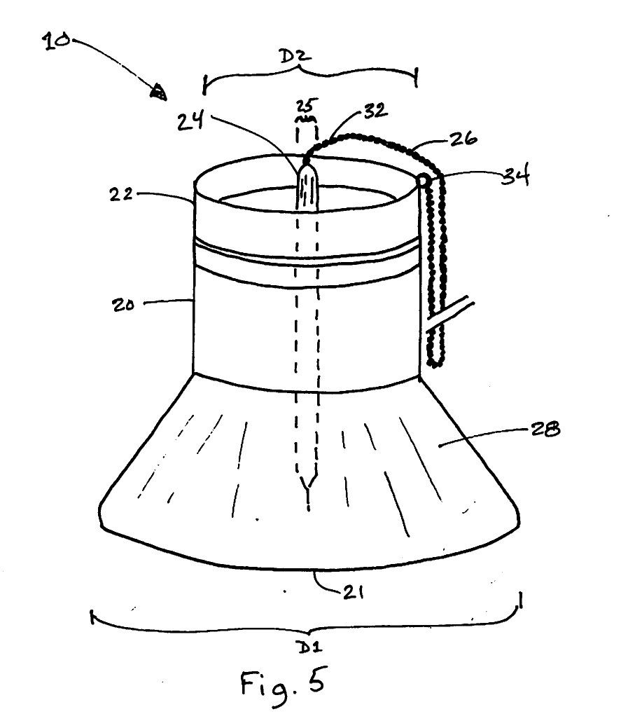

Referring now to FIG. 5, a further alternative embodiment is shown. The vessel 20, comprises bottom 21 that is enlarged, having a diameter D1 that is greater than the diameter D2 of the cover, thus providing a more stable base for the apparatus 10.

Inasmuch as the preceding disclosure presents the best mode devised by the inventor for practicing the invention and is intended to enable one skilled in the pertinent art to carry it out, it is apparent that methods incorporating modifications and variations will be obvious to those skilled in the art. As such, it should not be construed to be limited thereby but should include such aforementioned obvious variations and be limited only by the spirit and scope of the following claims.

I claim:

1. An apparatus for sanitizing a writing instrument comprising:
a vessel capable of containing an absorbent material, a sanitizing fluid and a writing instrument;
a cover, capable of being attached to said vessel and having an aperture through which a writing instrument may be received;
an absorbent material disposed within said vessel, wherein said absorbent material comprises a channel for receiving a writing instrument for sanitizing;
a sanitizing fluid capable of sanitizing a writing instrument, wherein said sanitizing fluid is disbursed within said absorbent material in said vessel; and
a writing instrument, attached to said apparatus with a connector, wherein said writing instrument can be inserted into said aperture and further inserted into said channel of said absorbent material wherein said writing instrument is sanitized by the sanitizing fluid disbursed within said absorbent material.

2. The apparatus of claim 1 wherein said vessel is constructed from a material resistant to degradation by said sanitizing fluid.

3. The apparatus of claim 1 wherein the aperture in said cover is concentric to the channel in said absorbent material.

4. The apparatus of claim 1 wherein the width of said channel in said absorbent material is smaller than the width of said writing instrument.

5. The apparatus of claim 1 wherein said absorbent material is selected from the group consisting of: sponge, cloth, natural material, synthetic material, wool, grass, hemp, sisal, straw, bamboo, cotton, linen, flax, jute, felt, casein fiber, silk, velvet, taffeta, lyocell, glass fiber, polyester, polyurethane, aramid, acrylic, acetate, nylon, spandex, olefin fiber, carbon fiber, burlap, and canvas.

6. The apparatus of claim 1 wherein said writing instrument is resistant to degradation by said sanitizing fluid.

7. The apparatus of claim 1 wherein said sanitizing fluid is hypoallergenic.

8. The apparatus of claim 1 wherein said writing instrument is selected from the group consisting of: a pen; a pencil; and a marker.

9. The apparatus of claim 1 wherein said connector is resistant to degradation by said sanitizing fluid.

10. The apparatus of claim 1 wherein said connector is selected from the group consisting of: a chain; a rope; a string; straps; webbing; a cord; a cable; a monofilament line; a woven line, and a string.

11. The apparatus of claim 1 wherein the bottom of said vessel comprises a non-slip surface to maintain the position of the apparatus on a substrate.

12. The apparatus of claim 1 wherein said vessel comprises a weight to maintain the position of the apparatus on a substrate.

13. The apparatus of claim 1 wherein said vessel comprises at least one suction cup to maintain the position of the apparatus on a substrate.

14. An apparatus for sanitizing a writing instrument comprising:
a vessel capable of containing an absorbent material, a sanitizing fluid and a writing instrument;
a cover, capable of being attached to said vessel and having an aperture disposed substantially in the center of said cover through which a writing instrument may be received;
an absorbent material disposed within said vessel, wherein said absorbent material comprises a channel, disposed within said absorbent material substantially concentric to said aperture in said cover, for receiving a writing instrument for sanitizing;
a sanitizing fluid capable of sanitizing a writing instrument, wherein said sanitizing fluid is disbursed within said absorbent material in said vessel; and
a writing instrument, attached to said apparatus with a connector, wherein said writing instrument is resistant to degradation by said sanitizing fluid and wherein said writing instrument can be inserted into said aperture and further inserted into said channel of said absorbent material and wherein said writing instrument is sanitized by the sanitizing fluid disbursed within said absorbent material.

15. The apparatus of claim 14 wherein the width of said channel in said absorbent material is smaller than the width of said writing instrument.

16. The apparatus of claim 14 wherein said absorbent material is selected from the group consisting of: sponge, cloth, natural material, synthetic material, wool, grass, hemp, sisal, straw, bamboo, cotton, linen, flax, jute, felt, casein fiber, silk, velvet, taffeta, lyocell, glass fiber, polyester, polyurethane, aramid, acrylic, acetate, nylon, spandex, olefin fiber, carbon fiber, burlap, and canvas.

17. The apparatus of claim 14 wherein said sanitizing fluid is hypoallergenic.

18. The apparatus of claim 14 wherein said connector is selected from the group consisting of: a chain; a rope; a string; straps; webbing; a cord; a cable; a monofilament line; a woven line, and a string.

19. The apparatus of claim 14 wherein the bottom of said vessel comprises a means to maintain the position of the apparatus on a substrate.

20. An apparatus for sanitizing a writing instrument comprising:
a vessel capable of containing an absorbent material, a sanitizing fluid and a writing instrument;
a cover, capable of being attached to said vessel and having an aperture disposed substantially in the center of said cover through which a writing instrument may be received;
an absorbent material disposed within said vessel, wherein said absorbent material comprises a channel, disposed within said absorbent material substantially concentric to said aperture in said cover, for receiving a writing instrument for sanitizing;
a sanitizing fluid capable of sanitizing a writing instrument, wherein said sanitizing fluid is disbursed within said absorbent material in said vessel;
a writing instrument, wherein said writing instrument is resistant to degradation by said sanitizing fluid and wherein said writing instrument can be inserted into said aperture and further inserted into said channel of said absorbent material and wherein said writing instrument is sanitized by the sanitizing fluid disbursed within said absorbent material and wherein the width of said writing instrument is larger than the width of said channel of said absorbent material; and
a connector for connecting said writing instrument to said apparatus wherein said connector is attached at a first end to said apparatus and attached at a second end to said writing instrument.
